SEQUENCE LISTING (2 sequences)

SEQ ID NO: 1 - DNA, 18 bp, Homo sapiens
gctgtggtggagaaactg

SEQ ID NO: 2 - DNA, 27 bp, Homo sapiens
tacacagataccacatttagcaggaac

(19) United States
(12) Patent Application Publication
(10) Pub. No.: US 2012/0184452 A1
(43) Pub. Date: Jul. 19, 2012

(54) METHODS FOR DIAGNOSING FOLLICULAR THYROID CANCER

(75) Inventors: Maria del Carmen Pastoriza Rodriguez, Santiago de Compostela (ES); Maria del Carmen Carneiro Freire, (US); José Manuel Cameselle-Teijeiro, Santiago de Compostela (ES); Fernando Dominguez Puente, (US); Anxo Vidal Figueroa, (US)

(73) Assignees: Universidade de Santiago de Compostela, Santiago de Compostela (ES); Fundacion Pedro Barrie de la Maza, Conde De Fenosa, A Coruna (ES); Fundacion Publica Galega de Medicina Xenomica, Santiago de Compostela (ES)

(21) Appl. No.: 13/344,749

(22) Filed: Jan. 6, 2012

Related U.S. Application Data
(60) Provisional application No. 61/432,316, filed on Jan. 13, 2011.

(51) Int. Cl. C40B 30/04; G01N 33/566; C12Q 1/68

(52) U.S. Cl. 506/9; 435/6.12; 436/501; 435/7.92

(57) ABSTRACT

Methods for diagnosing follicular thyroid cancer, providing a prognosis for follicular thyroid cancer, and monitoring treatment of follicular thyroid cancer, using biomarkers that are differentially expressed in follicular thyroid cancer are provided.

### RELATED APPLICATION

This application claims the benefit under 35 U.S.C. §119(e) of U.S. provisional application Ser. No. 61/432,316, filed Jan. 13, 2011, the disclosure of which is incorporated by reference herein in its entirety.

### BACKGROUND OF THE INVENTION

Cancers that arise from the thyroid gland are the most common endocrine malignancy. Moreover, incidence of thyroid cancers had increased over the last decades (Hodgson et al, 2004). In Spain, incidence is especially high in the northwest part of the country (Lope et al, 2006).

Iodide deficiency, smoking, a history of head and neck radiation, female gender, familial predisposition and increasing age are the principal risk factors for thyroid nodules (American Cancer Society, www.cancer.org).

The thyroid gland is located under the thyroid cartilage in the front part of the neck. It contains mainly two types of endocrine cells: thyroid follicular cells and C cells (also called parafollicular cells). Less abundant cells in the thyroid gland include immune system cells and supportive cells. Several types of tumors can develop in the thyroid gland, depending of what cell of the mentioned is the source of the alteration, and this becomes a very important issue, since it not only determines how serious the cancer is, but also what kind of treatment is needed.

Most of the tumors affecting the thyroid gland are benign (non-cancerous), but there is also a set of malignant tumors that not only will alter the correct function of the gland, but also can spread into the nearby tissue and to other parts of the body. Currently benign and malignant thyroid tumors are distinguished and assigned into specific subtypes based on a histological classification (Eszlinger and Paschke, 2010).

The application of the current World Health Organization (WHO) classification in clinical routine is impeded by high interobserver variability, that is most pronounced for follicular-patterned tumors. As an example, a study carried out by Franc et al, the level of agreement among five pathologists initial diagnosis and the final consensus diagnosis has been reported as 37% in the mean, and even the interobserver agreement for defined criteria like capsular invasion and vascular invasion was very low (only 27 and 20%, respectively) (Franc et al, 2003).

Fine needle aspiration biopsy (FNAB) has developed as the method of election for guiding the clinical management of patients with thyroid nodules. However, the application of FNAB to distinguish benign follicular nodules from follicular carcinomas is also problematic because the criteria for distinguishing between them are dependent on the presence of capsular or vascular invasion on formal pathological evaluation that can not be determined by FNAB smears (Faquim, 2009). Benign lesions (e.g., partly encapsulated hyperplastic nodules, pseudoinvasion after fine needle aspiration), and malignancies (especially the follicular variant of papillary thyroid carcinoma) have been sometimes misinterpreted as follicular carcinoma. Unfortunately, while FNAB as a screening test is highly sensitive, it lacks specificity. Approximately 15-30% of aspirates diagnosed as “suspicious for a follicular neoplasm” are actually carcinomas while the remaining 70-85% of nodules are benign. As a result, patients with this diagnosis are typically taken to the operating room for a thyroid lobectomy. If the final pathologic reading is carcinoma, most patients return to the operating room for completion thyroidectomy in anticipation of radioactive iodine ablation. The accurate, preoperative diagnosis of follicular lesions represents a clear diagnostic void that results in many unnecessary thyroid surgeries.

### SUMMARY OF THE INVENTION

In recent years, an increased number of immunohistological markers have being tested but few have entered routine use as many of them show prominent overlap between follicular adenomas (FA) and differentiated thyroid carcinomas (Faggiano et al, 2007). The discovery of point mutations and chromosomal rearrangements for about two thirds of papillary carcinomas (PC) and follicular thyroid carcinomas (FTC) initially generated new perspectives for the classification of thyroid tumors. However, it soon became apparent that the incidence of the different somatic mutations in FTCs and PCs varied from study to study (Kondo et al, 2006).

Recently, the use of gene expression signatures has emerged as a new tool to attempt thyroid tumors classification. However, despite the fact that several microarrays studies have revealed changes on the expression levels for certain genes that associated with a particular thyroid tumor type, none of then was proven to be an ideal single marker. Currently, an alternative approach that aims at identifying the minimal number of discriminating genes appears more promising for diagnostic purposes (Mazzanti et al, 2004; Jarzab et al, 2005; Weber et al, 2005; Eszlinger et al, 2006; Foukakis et al, 2007).

According to one aspect of the invention, methods of diagnosing follicular thyroid cancer in a human are provided. The methods include contacting a biological sample from a subject with reagents that specifically bind to a panel of biomarkers comprising ABI3BP and ANGPT2, and determining whether the biomarkers are differentially expressed in the sample relative to a control; thereby diagnosing follicular thyroid cancer.

In some embodiments, the panel of biomarkers includes EPHB1, ABI3BP and ANGPT2 or EPHB1, GPM6A, ABI3BP and ANGPT2. In some embodiments, the panel of biomarkers consists of (1) ABI3BP and ANGPT2; (2) EPHB1, ABI3BP and ANGPT2; or (3) EPHB1, GPM6A, ABI3BP and ANGPT2. In some embodiments, the sample is a biopsy such as a fine needle aspiration biopsy.

In some embodiments, the reagent includes or is a nucleic acid, such as an oligonucleotide or an RT PCR primer set. In other embodiments, the reagent includes or is an antibody or an antigen-binding fragment thereof, such as a monoclonal antibody.

In some embodiments, the expression of ANGPT2 is upregulated relative to a control and the expression of EPHB1, GPM6A and/or ABI3BP is downregulated relative to a control.

In some embodiments, the differential expression in the sample relative to the control is at least 1.8 fold. In some embodiments, the control is a level of expression determined in a non-malignant thyroid tissue sample.

According to another aspect of the invention, methods of determining prognosis of follicular thyroid cancer in a human are provided. The methods include contacting a biological sample from a subject with reagents that specifically bind to a panel of biomarkers comprising ABI3BP and ANGPT2, and determining whether the biomarkers are differentially expressed in the sample relative to a control; thereby providing a prognosis for the human.

In some embodiments, the panel of biomarkers includes EPHB1, ABI3BP and ANGPT2 or EPHB1, GPM6A, ABI3BP and ANGPT2. In some embodiments, the panel of biomarkers consists of (1) ABI3BP and ANGPT2; (2) EPHB1, ABI3BP and ANGPT2; or (3) EPHB1, GPM6A, ABI3BP and ANGPT2. In some embodiments, the sample is a biopsy such as a fine needle aspiration biopsy.

In some embodiments, the reagent includes or is a nucleic acid, such as an oligonucleotide or an RT PCR primer set. In other embodiments, the reagent includes or is an antibody or an antigen-binding fragment thereof, such as a monoclonal antibody.

In some embodiments, the expression of ANGPT2 is upregulated relative to a control and the expression of EPHB1, GPM6A and/or ABI3BP is downregulated relative to a control.

In some embodiments, the differential expression in the sample relative to the control is at least 1.8 fold. In some embodiments, the control is a level of expression determined in a non-malignant thyroid tissue sample.

According to another aspect of the invention, methods of monitoring treatment in a human are provided. The methods include obtaining biological samples from a subject before and after treatment for a follicular thyroid cancer, contacting the biological samples from the subject with reagents that specifically bind to a panel of biomarkers comprising ABI3BP and ANGPT2, and determining expression of the biomarkers in the samples. Reduction or elimination of differential expression of the panel of biomarkers relative to a control in the biological sample from a subject after treatment compared to the differential expression of the panel of biomarkers relative to a control in the biological sample from a subject before treatment indicates that the treatment for follicular thyroid cancer is successful.

In some embodiments, the panel of biomarkers includes EPHB1, ABI3BP and ANGPT2 or EPHB1, GPM6A, ABI3BP and ANGPT2. In some embodiments, the panel of biomarkers consists of (1) ABI3BP and ANGPT2; (2) EPHB1, ABI3BP and ANGPT2; or (3) EPHB1, GPM6A, ABI3BP and ANGPT2. In some embodiments, the samples are biopsies such as fine needle aspiration biopsies.

In some embodiments, the reagent includes or is a nucleic acid, such as an oligonucleotide or an RT PCR primer set. In other embodiments, the reagent includes or is an antibody or an antigen-binding fragment thereof, such as a monoclonal antibody.

In some embodiments, the expression of ANGPT2 is upregulated relative to a control and the expression of EPHB1, GPM6A and/or ABI3BP is downregulated relative to a control.

In some embodiments, the differential expression in the sample relative to the control is at least 1.8 fold. In some embodiments, the control is a level of expression determined in a non-malignant thyroid tissue sample.

According to another aspect of the invention, kits are provided. The kits include reagents that specifically bind to a panel of biomarkers comprising ABI3BP and ANGPT2. In some embodiments, the panel of biomarkers comprises EPHB1, ABI3BP and ANGPT2 or EPHB1, GPM6A, ABI3BP and ANGPT2. In some embodiments, the panel of biomarkers consists of (1) ABI3BP and ANGPT2; (2) EPHB1, ABI3BP and ANGPT2; or (3) EPHB1, GPM6A, ABI3BP and ANGPT2. In some embodiments, the samples are biopsies such as fine needle aspiration biopsies.

In some embodiments, the reagents include or are one or more nucleic acids, such as one or more oligonucleotides or one or more RT-PCR primers. In other embodiments, the reagents include or are one or more antibodies or antigen-binding fragments thereof, such as one or more monoclonal antibodies. In some embodiments, the reagents are detectably labeled or the kit further includes one or more detectable labels.

According to another aspect of the invention, methods of diagnosing thyroid adenocarcinoma in a human are provided. The methods include contacting a biological sample from a subject with reagents that specifically bind to full length FOLH1 and FOLH1 lacking exon 1 (PSM′), determining expression of FOLH1 and PSM′ in the sample, and determining a FOLH1/PSM′ ratio of expression; wherein a FOLH1/PSM′ ratio lower than 0.5 indicates that the subject has a thyroid adenocarcinoma.

In some embodiments, the sample is a biopsy such as a fine needle aspiration biopsy.

In some embodiments, the reagent includes or is a nucleic acid, such as an oligonucleotide or an RT PCR primer set. In other embodiments, the reagent includes or is an antibody or an antigen-binding fragment thereof, such as a monoclonal antibody.

According to another aspect of the invention, methods of determining prognosis of follicular thyroid cancer in a human are provided. The methods include contacting a biological sample from a subject with reagents that specifically bind to full length FOLH1 and FOLH1 lacking exon 1 (PSM′), determining expression of FOLH1 and PSM′ in the sample, and determining a FOLH1/PSM′ ratio of expression, wherein a FOLH1/PSM′ ratio lower than 0.5 indicates that the subject has a thyroid adenocarcinoma, thereby providing a prognosis for the human.

In some embodiments, the sample is a biopsy such as a fine needle aspiration biopsy.

In some embodiments, the reagent includes or is a nucleic acid, such as an oligonucleotide or an RT PCR primer set. In other embodiments, the reagent includes or is an antibody or an antigen-binding fragment thereof, such as a monoclonal antibody.

According to another aspect of the invention, methods of monitoring treatment in a human are provided. The methods include obtaining biological samples from a subject before and after treatment for a follicular thyroid cancer, contacting the biological samples from the subject with reagents that specifically bind to full length FOLH1 and FOLH1 lacking exon 1 (PSM′), determining expression of FOLH1 and PSM′ in the biological samples, and determining FOLH1/PSM′ ratios of expression, wherein an increase in the FOLH1/PSM′ ratio in the biological sample from a subject after treatment relative to the FOLH1/PSM′ ratio in the biological sample from a subject before treatment indicates that the treatment for follicular thyroid cancer is successful.

In some embodiments, the sample is a biopsy such as a fine needle aspiration biopsy.

In some embodiments, the reagent includes or is a nucleic acid, such as an oligonucleotide or an RT PCR primer set. In other embodiments, the reagent includes or is an antibody or an antigen-binding fragment thereof, such as a monoclonal antibody.

According to another aspect of the invention, kits are provided. The kits include reagents that specifically bind to FOLH1 and FOLH1 lacking exon 1 (PSM′).

In some embodiments, the reagents include or are one or more nucleic acids, such as one or more oligonucleotides or one or more RT-PCR primers. In other embodiments, the reagents include or are one or more antibodies or antigen-binding fragments thereof, such as one or more monoclonal antibodies. In some embodiments, the reagents are detectably labeled or the kit further includes one or more detectable labels.

These and other aspects of the invention will be described in further detail in connection with the detailed description of the invention.

### BRIEF DESCRIPTION OF THE DRAWINGS

FIG. 1 shows a schematic representation of sample processing from RNA extration to microarray hybridization.

FIG. 2 shows a schematic representation of workflow for gene-level and exon-level analysis.

FIG. 3 shows correlation between microarray and qPCR results.

FIG. 4 shows differential expression of FOLH1 exon 1. The signals detected from probes in an array covering the FOLH1 gene are shown. Signals from normal thyroid, adenomas and carcinomas obtained from the different probes (bottom rectangles) are always equivalent, except for the case of probe #3372937. Normal thyroid (bottom line at probe #3372937); adenomas (middle line at probe #3372937); carcinomas (top line at probe #3372937).

FIG. 5 shows FOLH1 (PSMA) mRNA splice variants and protein isoforms translated from difference splice variants (Schmittgen et al, 2003; Mlcochová et al, 2009). Patterned boxes show differences in amino acid sequence between the protein isoforms. IN=intracellular domain; TM=transmembrane domain; EXT=extracellular domain; AA=amino acids.

FIG. 6 shows analysis by qPCR of the FOLH1/PSM′ratio for normal (“N”; n=9), follicular adenomas (“FA”; n=17) and follicular carcinomas (“FC”; n=22) thyroid samples. One-way ANOVA; p=0.0003.

FIG. 7 shows a schematic diagram with the different sample sets and techniques used in this study.

### DETAILED DESCRIPTION OF THE INVENTION

Panels of two genes (ABI3BP and ANGPT2), three genes (EPHB1, ABI3BP and ANGPT2), four genes (EPHB1, GPM6A, ABI3BP and ANGPT2) and 42 genes (see Table 2) have been identified that show differential expression in benign and malignant thyroid tumors, as determined by microarray analysis and show an unexpected increase in predictive value when combined. qRT-PCR was used to confirm the microarray expression data for the three and four gene panels and these results confirmed that the three or four genes can be used as a panel of biomarkers for diagnosis and/or prognosis of thyroid cancer and for selecting or monitoring treatment of thyroid cancer. All of the genes in the two, three and four gene biomarker panels are independent diagnostic markers for distinguishing benign from malignant thyroid neoplasms. The combination of these two biomarkers, three biomarkers, four biomarkers, or 42 biomarkers provides and greater predictive value than any of the individual biomarkers alone.

In addition, an unexpected correlation between a ratio of expression of FOLH1/PSM′ (a short form of FOLH1 lacking exon 1) that is less than 0.5 and thyroid adenocarcinoma has been found. This ratio can be used for diagnosis and/or prognosis of thyroid adenocarcinoma and for selecting or monitoring treatment of thyroid adenocarcinoma.

EPHB1 is Ephrin receptor B1 (nucleotide sequence NM_{—}004441; polypeptide sequence NP_{—}004432). GPM6A is glycoprotein M6A (nucleotide sequence NM_{—}005277; polypeptide sequence NP_{—}005268). ANGPT2 is angiopoietin 2, also known as Ang2 (nucleotide sequence NM_{—}001118887; polypeptide sequence NP_{—}001112359). ABI3BP is ABI3-binding protein, also known as ABI family, member 3, (NESH) binding protein, and TARSH (nucleotide sequence NM_{—}015429; polypeptide sequence NP_{—}056244). Identification of additional genes in the 42 gene set is provided in Table 2. FOLH1 is folate hydrolase, also known as prostate-specific membrane antigen 1 (nucleotide sequence NM_{—}001014986; polypeptide sequence NP_{—}001014986).

The present invention provides methods of predicting, diagnosing or providing prognosis of thyroid cancer, or monitoring treatment of thyroid cancer, by detecting the expression of biomarkers differentially expressed in thyroid cancer. Prediction and diagnosis involve determining the level of a panel of thyroid cancer biomarker polynucleotides or the corresponding polypeptides in a patient or patient sample and then comparing the level to a baseline or range or control. Typically, the baseline value is representative of levels of the polynucleotide or nucleic acid in a healthy person not suffering from, or destined to develop, thyroid cancer, as measured using a biological sample such as a thyroid biopsy or a sample of a bodily fluid. Variation of levels of a polynucleotide or corresponding polypeptides of the invention from the baseline range (either up or down) indicates that the patient has an increased risk of developing thyroid cancer or an increased risk of its recurrence. For distinguishing between malignant and benign thyroid neoplasms, a panel including or consisting of two biomarkers (ABI3BP and ANGPT2), three biomarkers (EPHB1, ABI3BP and ANGPT2), four biomarkers (EPHB1, GPM6A, ABI3BP and ANGPT2) or 42 biomarkers (see Table 2) is used. Alternatively, a ratio of FOLH1/PSM′ is used for distinguishing between malignant and benign thyroid neoplasms.

As used herein, the term “diagnosis” refers to distinguishing between malignant and benign thyroid neoplasms. As used herein, the term “prognosis” refers to a prediction of the probable course and outcome of the thyroid cancer. In some embodiments, the thyroid cancer is a follicular thyroid cancer, such as a thyroid follicular adenocarcinoma.

Thus in some embodiments, methods of diagnosing thyroid cancer in a subject are provided. The methods include steps of contacting a biological sample obtained from the subject with reagents that specifically bind to a panel of biomarkers comprising or consisting of two biomarkers (ABI3BP and ANGPT2), three biomarkers (EPHB1, ABI3BP and ANGPT2), four biomarkers (EPHB1, GPM6A, ABI3BP and ANGPT2) or 42 biomarkers (see Table 2), and determining whether or not the biomarker is differentially expressed in the sample, optionally relative to a control, thereby providing a diagnosis for thyroid cancer.

Additional methods of diagnosing thyroid cancer in a subject are provided. Such methods include steps of contacting a biological sample obtained from the subject with reagents that specifically bind to biomarkers FOLH1 and PSM′, determining the expression of the FOLH1 and PSM′ in the sample, optionally relative to a control, and determining a ratio or FOLH1/PSM′, which ratio provides a diagnosis for thyroid cancer.

In other embodiments, methods of determining prognosis of thyroid cancer in a human are provided. The methods include steps of contacting a biological sample obtained from the subject with reagents that specifically bind to a panel of biomarkers comprising or consisting of two biomarkers (ABI3BP and ANGPT2), three biomarkers (EPHB1, ABI3BP and ANGPT2), four biomarkers (EPHB1, GPM6A, ABI3BP and ANGPT2) or 42 biomarkers (see Table 2), and determining whether or not the biomarker is differentially expressed in the sample, optionally relative to a control, thereby providing a prognosis for the human.

Additional methods of determining prognosis of thyroid cancer in a subject are provided. Such methods include steps of contacting a biological sample obtained from the subject with reagents that specifically bind to biomarkers FOLH1 and PSM′, determining the expression of the FOLH1 and PSM′ in the sample, optionally relative to a control, and determining a ratio or FOLH1/PSM′, which ratio provides a prognosis for the human.

Still other embodiments provide methods of monitoring treatment of thyroid cancer in a human. The methods include steps of contacting a biological sample from a subject being treated for thyroid cancer with reagents that specifically bind to a panel of biomarkers comprising or consisting of two biomarkers (ABI3BP and ANGPT2), three biomarkers (EPHB1, ABI3BP and ANGPT2), four biomarkers (EPHB1, GPM6A, ABI3BP and ANGPT2) or 42 biomarkers (see Table 2), and determining whether the biomarkers are differentially expressed in the sample, optionally relative to a control. Reduction or elimination of differential expression of the panel of biomarkers indicates that the treatment for thyroid cancer is successful. Conversely, an increase in the differential expression of the panel of biomarkers indicates that the treatment for thyroid cancer is not successful.

Additional methods of monitoring treatment of thyroid cancer in a human are provided. Such methods include steps of contacting a biological sample obtained from the subject with reagents that specifically bind to biomarkers FOLH1 and PSM′, determining the expression of the FOLH1 and PSM′ in the sample, optionally relative to a control, and determining a ratio or FOLH1/PSM′, which ratio provides a prognosis for the human. An increase in the FOLH1/PSM′ ratio to above 0.5 or higher indicates that the treatment for thyroid cancer is successful. Conversely, a lowering of the FOLH1/PSM′ ratio to below 0.5 (or lower) indicates that the treatment for thyroid cancer is not successful.

In some embodiments, the foregoing methods distinguish low risk and high risk thyroid cancers. By determining whether or not the biomarkers are differentially expressed in the sample, the risk associated with the thyroid cancer can be determined. This distinction between low risk and high risk thyroid cancers is useful in diagnosis, prognosis and in monitoring treatment.

The invention also comprises kits that can be used in performing any of the methods described herein, which includes reagents that specifically bind to the genes in the biomarker panels described herein [two genes (ABI3BP and ANGPT2), three genes (EPHB1, ABI3BP and ANGPT2), four genes (EPHB1, GPM6A, ABI3BP and ANGPT2) or 42 genes (see Table 2)], or to FOLH1 and PSM′ to enable determination of a ratio of FOLH1/PSM′ expression. The kits can be used to analyze patient samples such as biopsies for diagnosis, predicting thyroid cancer patient outcome (prognosis), and for selecting or monitoring treatment of the patient. The kit optionally includes reagents for amplification of the biomarkers from a biological sample. The kit optionally includes nucleic acids and/or other reagents for analyzing nucleic acid expression, such as by performing RT-PCR, or microarray analysis. The kit optionally or alternatively includes reagents for detecting biomarker polypeptides in a biological sample, such as antibodies or antigen-binding fragments thereof that specifically bind to polypeptides encoded by the genes in the biomarker panels described herein [two genes (ABI3BP and ANGPT2), three genes (EPHB1, ABI3BP and ANGPT2), four genes (EPHB1, GPM6A, ABI3BP and ANGPT2) or 42 genes (see Table 2)], or that specifically bind to FOLH1 and PSM′ polypeptides.

The two most common types of malignant thyroid tumors are the papillary carcinoma and the follicular carcinoma, both derived from thyroid follicular cells. Several different variants (subtypes) of papillary carcinoma can be recognized under the microscope. Of these, the follicular variant (also called mixed papillary-follicular variant) occurs most often. Papillary carcinomas often spread to the lymph nodes in the neck, but in most of the cases it can be successfully treated. Follicular carcinomas however, usually do not spread to the lymph nodes, but some of them can spread to other parts of the body. Less abundant are medullary thyroid carcinoma, which evolves from thyroid C cells, anaplastic carcinoma, a rare and very aggressive form of thyroid cancer, and thyroid lymphoma (American Cancer Society, www.cancer.org).

The term “biomarker” as used herein refers to a molecule (nucleic acid or the polypeptide encoded by the nucleic acid) that is expressed by a malignant thyroid cancer cell at a different level in comparison to a non-cancer cell or a non-malignant cancer cell, and which is useful for the diagnosis of cancer, for providing a prognosis, and/or for monitoring treatment of the cancer in a subject. It will be understood by persons skilled in the art that biomarkers may be used in combination with other biomarkers or tests for any of the uses, e.g., prediction, diagnosis, prognosis of cancer, or monitoring treatment, as disclosed herein.

A “biological sample” as used herein includes sections of tissues such as biopsy and autopsy samples, and frozen sections taken for histologic purposes. Such samples include thyroid tissue and may also include other tissue or cells, such as surrounding tissue. A biological sample is typically obtained from a human but may also be obtained from other mammals.

A biological sample from a subject, such as a human subject, can be obtained directly from the subject, for example by tumor biopsy. A biological sample also can be obtained from a third party, for example, a physician or hospital performing the biopsy procedure on the subject, or a third party that handles, stores, archives, and/or processes the sample. Methods for performing tumor biopsies are well known to those of skill in the art.

A “biopsy” refers to the process of removing a tissue sample for diagnostic or prognostic evaluation, and to the tissue specimen itself, which can be used as a biological sample in the methods described herein. Any biopsy technique known in the art can be applied in the diagnostic, prognostic and monitoring methods of the invention. As known to those skilled in the art, the biopsy technique applied will depend on the tissue type to be evaluated (e.g., thyroid etc.), the size and type of the tumor, among other factors. Representative biopsy techniques include, but are not limited to, excisional biopsy, incisional biopsy, needle biopsy, such as a fine-needle aspiration biopsy, and surgical biopsy. Biopsy techniques are discussed, for example, in Harrison's Principles of Internal Medicine, Kasper, et al., eds., 16th ed., 2005, Chapter 70, and throughout Part V.

The term “differentially expressed” or “differentially regulated” refers generally to a protein or nucleic acid that is overexpressed (upregulated) or underexpressed (downregulated) in one sample compared to at least one other sample, generally in a cancer patient, in comparison to a patient without cancer, or in a cancer cell in comparison to a non-cancer cell, or in a malignant tumor cell in comparison to a non-malignant tumor cell, or as compared to a control.

In some embodiments, the level of expression of one or more of the biomarkers described herein in a sample being investigated is at least about 5%, about 10%, 10-50%, about 20%, about 30%, about 40%, about 50%, 50-100%, about 60%, about 70%, about 80%, about 90%, about 100%, 100-150%, about 150%, 150-200%, about 200%, 200-250%, about 250%, 250-500%, about 300%, about 400%, about 500%, 500-1000%, about 1000%, 1000-2500%, about 1500%, about 2000%, about 2500%, about 3000%, about 4000%, about 5000%, 5000%-10000%, about 6000%, about 7000%, about 8000%, about 9000%, or about 10000%, or more, different than the level of the one or more of the biomarkers described herein observed in negative control tissue or cell sample, indicating differential expression in the sample being examined. In some embodiments, the expression is greater by such amounts that the control, indicating increased expression in the sample being examined. In some embodiments, the expression of the one or more of the biomarkers described herein in a sample differs from (e.g., is greater than) a control by at least about 1.8 fold.

The terms “overexpress,” “overexpression” or “overexpressed” interchangeably refer to a protein or nucleic acid (RNA) that is transcribed or translated at a detectably greater level, usually in a cancer cell in comparison to a non-cancer cell, or in a malignant tumor cell in comparison to a non-malignant tumor cell, or as compared to a control. The term includes overexpression due to transcription, post transcriptional processing, translation, post-translational processing, and RNA and protein stability. Overexpression can be detected using conventional techniques for detecting mRNA (i.e., RT-PCR, PCR, hybridization) or proteins (i.e., ELISA, immunohistochemical techniques). Overexpression can be 10%, 20%, 30%, 40%, 50%, 60%, 70%, 80%, 90% or more in a cancer cell, in comparison to a non-cancer cell, or in a malignant tumor cell in comparison to a non-malignant tumor cell, or as compared to a control. In certain instances, overexpression is at least about 1-fold, 1.1-fold, 1.2-fold, 1.3-fold, 1.4-fold, 1.5-fold, 1.6-fold, 1.7-fold, 1.8-fold, 1.9-fold, 2-fold, 2.2-fold, 2.3-fold, 2.4-fold, 2.5-fold, 2.6-fold, 2.8-fold, 3-fold, 4-fold or more higher levels of transcription or translation in a cancer cell in comparison to a non-cancer cell, or in a malignant tumor cell in comparison to a non-malignant tumor cell, or as compared to a control.

The terms “underexpress,” “underexpression” or “underexpressed” or “downregulated” interchangeably refer to a protein or nucleic acid that is transcribed or translated at a detectably lower level in a cancer cell in comparison to a non-cancer cell, or in a malignant tumor cell in comparison to a non-malignant tumor cell, or as compared to a control. The term includes underexpression due to transcription, post transcriptional processing, translation, post-translational processing, and RNA and protein stability, in a cancer cell in comparison to a non-cancer cell, or in a malignant tumor cell in comparison to a non-malignant tumor cell, or as compared to a control. Underexpression can be detected using conventional techniques for detecting mRNA (i.e., RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In certain instances, underexpression is 1-fold, 1.1-fold, 1.2-fold, 1.3-fold, 1.4-fold, 1.5-fold, 1.6-fold, 1.7-fold, 1.8-fold, 1.9-fold, 2-fold, 2.2-fold, 2.3-fold, 2.4-fold, 2.5-fold, 2.6-fold, 2.8-fold, 3-fold, 4-fold or more lower levels of transcription or translation in a cancer cell in comparison to a non-cancer cell, or in a malignant tumor cell in comparison to a non-malignant tumor cell, or as compared to a control.

“Nucleic acid” refers to polymers of deoxyribonucleotides or ribonucleotides in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

A particular nucleic acid sequence also implicitly encompasses “splice variants” and nucleic acid sequences encoding truncated forms of a protein. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. An example of this is the FOLH1 gene, which encodes full length FOLH1 protein and PSM′ protein, the latter of which lacks the amino acids encoded by exon 1 of FOLH1. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The phrase “stringent hybridization conditions” refers to conditions under which a nucleic acid probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). The person of skill in the art is familiar with a variety of suitable stringent hybridization conditions and selection of suitable conditions for use in particular assays. Exemplary stringent hybridization conditions can be as following: 50% formamide, S×SSC, and 1% SDS, incubating at 42° C., or S×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

PCR protocols and guidelines for low and high stringency amplification reactions are well known in the prior art, and are provided, e.g., in Innis et al. (1990) PCR Protocols, A Guide to Methods and Applications, Academic Press, Inc. N.Y.).

The nucleic acids of the biomarker genes of this invention or their encoded polypeptides refer to all forms of nucleic acids (e.g., gene, pre-mRNA, mRNA) or proteins, their polymorphic variants, alleles, mutants, and interspecies homologs that (as applicable to nucleic acid or protein): (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, and immunogenic fragments thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. Alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)). Preferred examples of an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, e.g., with default parameters, to determine percent sequence identity for the nucleic acids and proteins of the invention. Software for performing BLAST analyses is publicly available through the National Center for Biotechnology Information (www.ncbi.nlm.nih.gov/).

Analysis of nucleic acid biomarkers and variants thereof can be performed using techniques known in the art including, without limitation, microarrays, polymerase chain reaction (PCR)-based analysis, sequence analysis, and electrophoretic analysis. A non-limiting example of a PCR-based analysis includes a TAQMAN® allelic discrimination assay available from Applied Biosystems. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry (MALDI-TOF/MS, and sequencing by hybridization.

Nucleic acid binding molecules such as probes, oligonucleotides, oligonucleotide arrays, and primers can be used in assays to detect differential RNA expression in patient samples, e.g., RT-PCR. Nucleic acid reagents that bind to selected biomarkers can be prepared according to methods known to those of skill in the art or purchased commercially.

General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

The terms “polypeptide,” “peptide” and “protein” are used interchangeably herein to refer to a polymer of amino acid residues. The terms apply to amino acid polymers in which one or more amino acid residues is an artificial chemical mimetic of a corresponding naturally occurring amino acid, as well as to naturally occurring amino acid polymers and non-naturally occurring amino acid polymers.

“Antibody” refers to a polypeptide comprising a framework region from an immunoglobulin gene or fragments thereof that specifically binds and recognizes an antigen. As is well-known in the art, only a small portion of an antibody molecule, the paratope, is involved in the binding of the antibody to its epitope (see, in general, Clark, W.R., 1986, The Experimental Foundations of Modern Immunology, Wiley & Sons, Inc., New York; Roitt, I., 1991, Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

Thus, as will be apparent to one of ordinary skill in the art, the present invention also provides for F(ab′)2, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies, domain antibodies and camelid heavy chain antibodies.

An antibody in some embodiments is conjugated to an “effector” moiety. The effector moiety can be any number of molecules, including detectable labels such as radioactive labels or fluorescent labels, or can be a therapeutic moiety. A “label” or a “detectable moiety” is a composition detectable by spectroscopic, photochemical, biochemical, immunochemical, chemical, or other physical means. Thus antibodies may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types.

Antibody reagents can be used in assays to detect expression levels of the biomarkers of the invention in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques well known in the art, including competitive and non-competitive immunoassays, can be used. The term immunoassay encompasses techniques including, without limitation, enzyme immunoassays (EIA) such as enzyme-linked immunosorbent assay (ELISA), enzyme multiplied immunoassay technique (EMIT), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997).

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), rhodamine, Texas red, tetrarhodamine isothiocynate (TRITC), Cy3, Cy5, etc.), fluorescent markers (e.g., green fluorescent protein (GFP), phycoerythrin, etc.), enzymes (e.g., luciferase, horseradish peroxidase, alkaline phosphatase, etc.), nanoparticles, biotin, digoxigenin, and the like.

Specific immunological binding of the antibody to nucleic acids can be detected directly or indirectly. Direct labels include fluorescent or luminescent tags, metals, dyes, radionuclides, and the like, attached to the antibody. A chemiluminescence assay using a chemiluminescent antibody specific for the nucleic acid is suitable for sensitive, non-radioactive detection of protein levels. An antibody labeled with fluorochrome is also suitable.

A signal from the direct or indirect label can be analyzed, for example, using a spectrophotometer to detect color from a chromogenic substrate; a radiation counter to detect radiation; or a fluorometer to detect fluorescence in the presence of light of a certain wavelength.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

The phrase “specifically (or selectively) binds” when referring to a protein, nucleic acid, or antibody refers to a binding reaction that is determinative of the presence of the protein or nucleic acid, such as the differentially expressed biomarkers of the present invention, often in a heterogeneous population of proteins or nucleic acids and other biological molecules, such as a biological sample.

In some embodiments, an expression level, absolute or relative, of one or more of the biomarkers described herein in a biological sample is compared to a reference or control level. In some embodiments, if the expression level of the biomarkers differs from (e.g., is higher than) a reference or control level, then the subject from whom the sample was obtained is diagnosed as having a follicular thyroid cancer, such as a thyroid adenocarcinoma, and/or a bad prognosis. In other embodiments, expression levels of FOLH1 and PSM′ are determined in a sample and the FOLH1/PSM′ ratio is determined, wherein a ratio less than 0.5 indicates the presence of thyroid adenocarcinoma and a bad prognosis for the subject from whom the sample was obtained. In other embodiments, the expression levels of the four gene biomarker panel or the FOLH1/PSM′ ratio can be used to monitor the response of the subject to treatment, wherein response to treatment is indicated by the expression levels of the biomarkers or the FOLH1/PSM′ ratio becoming less like that of a malignant tumor and more like that of a normal tissue or non-malignant tumor.

Determination of gene expression in a tumor can be effected via numerous methods known to those of skill in the art. In some embodiments, a biological sample, for example, a tumor biopsy, may be obtained and the presence or absence and/or a level of expression may be determined by detecting an expression product of one or more of the biomarkers described herein, for example a protein or RNA transcript. Suitable binding agents are well known in the art and include, but are not limited to, antibodies and antigen-binding fragments thereof, aptamers, adnectins, etc. Antibodies specifically binding the biomarkers described herein are also commercially available, for example: against GPM6A, polyclonal antibodies HPA017338 from Sigma-Aldrich (St. Louis, Mo.) or AP9341b from Abgent (San Diego, Calif.); against EPHB1: monoclonal antibody 3980S from Cell Signaling Technologies (Danvers, Mass.) or polyclonal antibody sc-28979 from Santa Cruz Biotechnologies (Santa Cruz, Calif.); against ABI3BP, monoclonal antibody H00025890-M15 from Novus Biologicals (Littleton, Colo.) or polyclonal antibody ab68612 from Abcam (Cambridge, Mass.); and against ANGPT2, monoclonal antibody CMA105 from Cell Sciences (Canton, Mass.) or polyclonal antibody PAB 12280 from Abnova (Neihu District, Taipei City, Taiwan).

In some embodiments, a level of expression of one or more of the biomarkers described herein in a sample is determined. In some embodiments, expression of the genes in the biomarker panels described herein [two biomarkers (ABI3BP and ANGPT2), three biomarkers (EPHB1, ABI3BP and ANGPT2), four biomarkers (EPHB1, GPM6A, ABI3BP and ANGPT2) or 42 biomarkers (see Table 2)] is determined. In other embodiments, expression of FOLH1 and PSM′ is determined. Methods such as microarrays, RT-PCR, northern blot, ELISA, western blot, and other methods employing specific binding as known to the person skilled in the art can be used to yield quantifiable data. The expression of the one or more of the biomarkers described herein can be determined as the average expression level of each biomarker within a population of cells, for example in a biopsy sample obtained from a subject.

A control or reference level, also referred to as baseline level, can be determined using standard methods known to those of skill in the art. In some embodiments the control or reference level is a negative control or reference level, for example a level found or expected to be found in a normal thyroid cell or normal thyroid tissue. Examples of methods for determining a control or reference level include, for example, determining a level of one or more of the biomarkers described herein in a thyroid cell or tissue from a subject not afflicted with thyroid cancer or a subject who has a thyroid adenoma but not a thyroid adenocarcinoma, or determining an average or mean level of one or more of the biomarkers described herein in such cells or tissues from a plurality of subjects. Alternatively, a level of one or more of the biomarkers described herein may be determined in non-malignant tissue of a subject who has a thyroid cancer (such as a follicular thyroid cancer, such as a thyroid adenocarcinoma), for example, in non-malignant tissue surrounding a malignant tumor. In some embodiments, a control or reference level may be a historical value, a theoretical value, or an empirical value.

The invention provides compositions, kits and integrated systems for practicing the assays described herein using antibodies specific for the polypeptides or nucleic acids specific for the polynucleotides of the invention.

Kits for carrying out the diagnostic assays of the invention typically include a probe that comprises an antibody or nucleic acid sequence that specifically binds to polypeptides or polynucleotides of the invention, and a label for detecting the presence of the probe. The kits may include one or more antibodies that specifically bind to the proteins encoded by the biomarkers of the invention (or antigen-binding fragments thereof) or one or more nucleic acid sequences that specifically bind to biomarker polynucleotide sequences. In such embodiments, the antibodies can be provided in individual containers or compartments of a kit, or may be combined. The foregoing kits can include instructions or other printed material on how to use the various components of the kits for diagnostic, prognostic and/or monitoring purposes.

### EXAMPLES

This work is focused on follicular thyroid tumours, since despite of not being the most frequent form of thyroid tumours (those being the papillary) or the more aggressive (those being the anaplastic), they represent a diagnostic challenge in order to differentiate benign (adenomas) from malignant lesions (carcinomas). Correct diagnosis of these lesions is necessary to get the most appropriate treatment. Even though survival after resection (bilateral thyroidectomy) is high in these tumors (>90%), since thyroid hormones are essential for life, patients need to be under hormonal substitutive therapy for the rest of their life. For at least this reason, avoiding unnecessary resections will increase life quality for these patients.

Recent molecular analysis of follicular thyroid lesions suggests that follicular adenomas (FA) and follicular thyroid carcinomas (FTC) have characteristic microarray expression profiles that differentiate each of these lesions (Finley et al, 2004; Barden et al, 2003; Cerutti et al, 2004; Griffith et al, 2006). These studies have employed 3′IVT (3′ in vitro transcribed) or cDNA microarrays. While microarray analysis is difficult to translate into clinical use, however, these data can be used as a basis for development of more routine clinical tests.

This study takes advantage of the latest technology for genome wide gene expression quantitation, the Exon arrays. Historically, microarrays have interrogated the few hundred bases proximal to the 3′end of each gene, and used expression at the 3′end to approximate expression of the entire gene. This approach is compatible with the 3′-oligo (dT)-based priming and labelling assays and provides valuable insight into global gene expression. However, this approach assumes that the 3′-end of each gene is clearly defined, that each transcript has an intact poly-A tail and that the entire length of the gene is expressed as a single unit. These assumptions do not apply to all genes or samples. More than 60% of genes are known to be alternatively spliced, breaking with the biology dogma “one gene, one protein”. In these genes, a single pre-messenger RNA yields more than one type of RNA transcript, and each type of RNA transcript may lack one or more exons or parts of exons. This will determine at the end the generation of several different proteins that might differ in their functions. As many as 50% of disease-related point mutations may result in splice pattern changes and 20% of cancer-causing mutations can result in exons-skipping events. Unfortunately, classical 3′-expression microarrays do not discriminate between alternatively spliced transcripts that have identical 3′-ends. Transcripts lacking 3′-exon due to alternative splicing, non-polyadenylation (non poly-A tail), genomic deletions or other non-canonical genomic events, are not detected in 3′-based expression experiments.

These problems have been overcome recently by the development of the Exon array technology. Exon arrays offer a greater number of probes (short nucleotide sequences) along the entire length of each transcript compared to traditional 3′-IVT expression arrays (on average 40 vs. 11 probes per transcript). Moreover, probe-sets are not biased towards the 3′-end, since exons are distributed along the entire gene. Overall, exon arrays offer all the advantages of the traditional expression arrays, with the advantage of more and unbiased probes, besides the opportunity to perform analysis of differential exon expression.

### Materials and Methods

### Exon Arrays

The Exon array used in this project was the Human Exon 1.0 ST Array from Affymetrix. On this array, each gene is covered by 40 probes, with approximately four probes per exon. Multiple probes per exon enable “exon-level” analysis and allow one to distinguish between different isoforms of a gene. This exon-level analysis makes it possible to detect specific alterations in exon usage that may play a central role in disease mechanism and etiology.

### Samples: Source and Processing

Samples Source

Thyroid samples derived from patients at Hospital Clinico Universitario de Santiago (Santiago de Compostela, Spain) were collected immediately after surgical resection, snap frozen, and stored at −80° C. All samples were visually inspected on 5-μm hematoxylin and eosin-stained frozen sections by a pathologist.

Originally, 55 samples were obtained, being all normal functioning (normal secretion of thyroid hormones), with the exception of a group of hypersecreting tumours with TSHR (thyroid stimulating hormone receptor) mutations.

Sample Processing

Cryostat sections were disrupted using a Polytron homogenizer and total RNA was isolated from cryostat sections using RNeasy Mini Kit (Qiagen) or the RNA SpinII Kit (Macherey-Nagel) following the manufacturer's instructions. To assess RNA quality, all samples were analyzed using the Agilent 2100 Bioanalyzer and the RNA integrity number (RIN) was established. Only those samples with a RIN >7 were selected for microarray hybridization (Schroeder A, Mueller O, Stocker S, et al. The RIN: an RNA integrity number for assigning integrity values to RNA measurements. BMC Mol Biol 2006; 7: 3).

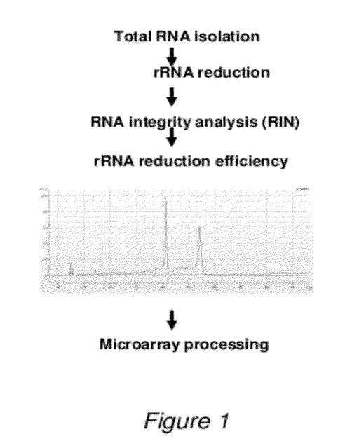

Total RNA samples (RIN >7) were then depleted of rRNA (ribosomal RNA) using the RiboMinus Kit (Invitrogen). A typical Bioanalyzer profile is shown below before (blue, upper trace with peaks) and after (red, lower trace) rRNA depletion, where two major peaks correspond to the two major rRNAs 28S and 18S in tissue. One microgram of total RNA was used to rRNA reduction, synthesis, fragmentation and labeling following the standard Affymetrix Whole-Transcript Sense Target-Labeling Assay protocol. Each thyroid sample was hybridized with an Affymetrix Human Exon 1.0 ST microarray. Background correction, normalization, probe summarization and data analysis was done with Partek Genomics Suite software using RMA algorithm. The entire process is depicted schematically in FIG. 1.

After quality controls, pre- and post-hybridization, 42 thyroid samples (from both males and females, all normal functioning) were included in the analysis, grouped as follows:
13 normal thyroid samples
12 follicular adenomas
17 follicular carcinomas (12 minimally invasive; 5 widely invasive).
   Microarray Data Acquisition and Preprocessing. Microarray Data Analysis.

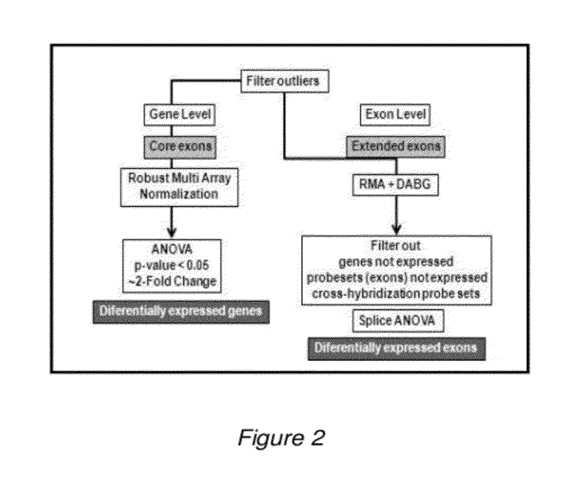

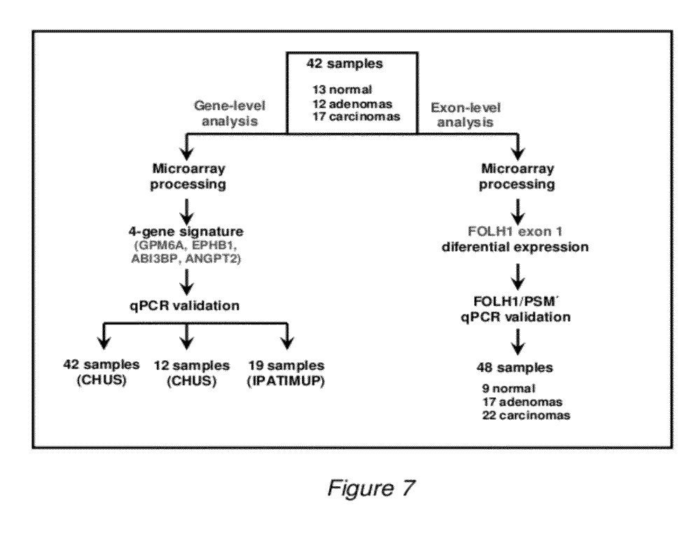

After microarray hybridization and data collection, CEL files were imported into Partek Genomics Suite software and preprocessed using the RMA (robust multiarray analysis) algorithm with the default parameters. A workflow of the data analysis is shown in FIG. 2. FIG. 7 shows a schematic diagram with the different sample sets and techniques used in this study.

### Gene-Level Analysis

Gene expression alterations were determined using Partek Genomics Suite software. The CEL files were imported and background correction, normalization and probe summarization was performed using RMA (robust multiarray analysis) algorithm. Principle Component Analysis (PCA) was performed (to assess hybridization quality).

The summarization and PCA analysis was followed by an analysis of variance (ANOVA) for the groups malignant (carcinoma) vs. non-malignant (adenoma plus normal tissue). We selected those genes that had p-value <0.05 and a fold-change >1.8. We used Principal Component Analysis and clustering analysis to reduce the gene list.

Quantitative PCR Analysis (qPCR).

Total RNA was isolated as described before and checked for integrity by using the Agilent 2100 bioanalyzer. Only samples with a RIN >4.1 where selected for the qPCR. From each sample, 500 ng were retrotranscribed by using the M-MLV retrotranscriptase (Invitrogen). qRT-PCR reactions were performed on an ABI PRISM 7300 HT Sequence Detection System (Applied Biosystems) using TaqMan® Gene Expression Assays (Applied Biosystems). Assay reference numbers are shown in Table 1. The ΔCt value for each sample was calculated by subtracting the mean of the Ct values for the housekeeping genes to the individual Ct value for each gene.

TABLE 1
TaqMan Gene Expression Assays

| TaqMan Probe Ref. | Gene Symbol | Description |
|---|---|---|
| Hs 01048042_m1 | ANGPT2 | Gene-signature |
| Hs 00227206_m1 | ABI3BP | Gene-signature |
| Hs 01009142_m1 | GPM6A | Gene-signature |
| Hs 00174725_m1 | EPHB1 | Gene-signature |
| Hs 99999903_m1 | ACTB | Housekeeping gene |
| Hs 99999905_m1 | GAPDH | Housekeeping gene |
| Hs 99999908_m1 | GUSB | Housekeeping gene |
| Hs 99999911_m1 | TFRC | Housekeeping gene |
| Hs 01020195_m1 | FOLH1 | Specific exon 1 |
| Hs 00379515_m1 | FOLH1 | External control |
| | | (exon boundary 4-5) |
| Custom TaqMan Gene | PSM′ | Specific PSM′ |
| Expression Assay | | transcript |

### DNA Sequencing.

To detect FOLH1 transcript variants, total RNA obtained from LNCaP cells was retrotranscribed as described above. A PCR reaction was performed with the following primers:

| | |
|---|---|
| forward | |
| 5′-GCTGTGGTGGAGAAACTG; | (SEQ ID NO: 1) |
| reverse | |
| 5′-TACACAGATACCACATTTAGCAGGAAC. | (SEQ ID NO: 2) |

PCR products were purified (Wizard SV Gel PCR Clean-up System, Promega), subcloned on a pGEM-T easy vector (Promega) and sequenced on an ABI 3730xl DNA Analyzer (Applied Biosystems).

### Example 1

### Identification of a Four-Gene Signature as Cancer (Malignant Vs Non-Malignant) Predictor

Gene-Level Analysis

After sample hybridization, data obtained from the arrays was processed as described in gene-level analysis, and transformed into a color-code image (not shown) that represents the expression levels of all genes that satisfy a previously established criteria (p<0.05, fold-change >1.8). An initial 42-gene signature that discriminates between carcinomas and non-carcinomas was determined. Based on this signature, almost all samples within each class are grouped together, meaning that it should be possible to classify an external sample as a non-malignant or as a FC by analyzing this 42-gene expression set.

TABLE 2
42-gene signature.

| Transcript ID | Gene | p-value | FoldChange |
|---|---|---|---|
| 3122489 | ANGPT2//angiopoietin 2 | 0.0012 | −2.5032 |
| 3150579 | ENPP2//ectonucleotide pyrophosphatase/phosphodiesterase 2 | 0.0171 | −2.4949 |
| 3250237 | HKDC1//hexokinase domain containing 1 | 0.0224 | −2.3042 |
| 2509900 | KIF5C//kinesin family member 5C | 0.0162 | −2.3042 |
| 3260586 | SCD//stearoyl-CoA desaturase (delta-9-desaturase) | 0.0147 | −2.2794 |
| 3305801 | SORCS1//sortilin-related VPS10 domain containing receptor 1 | 0.0432 | −2.0598 |
| 2536486 | (chr2: 242282185-242282284 (+), Len = 100) | 0.0172 | −2.0433 |
| 2967249 | BVES//blood vessel epicardial substance | 0.0174 | −2.0176 |
| 3592755 | SEMA6D//sema domain, transmembrane domain (TM), | 0.0197 | −2.0176 |
| | and cytoplasmic domain, (semaphorin) 6D | | |
| 3392888 | (chr11: 116657685-116658028 (−), Len = 344) | 0.0115 | −2.0171 |
| 3475764 | GPR81//G protein-coupled receptor 81 | 0.0418 | −1.9660 |
| 3671202 | CDH13//cadherin 13, H-cadherin (heart) | 0.0020 | −1.9596 |
| 2856995 | ESM1//endothelial cell-specific molecule 1 | 0.0147 | −1.9498 |
| 2598261 | FN1//fibronectin 1 | 0.0200 | −1.9330 |
| 3250990 | UNC5B//unc-5 homolog B (C. elegans) | 0.0086 | −1.9064 |
| 2862841 | GCNT4//glucosaminyl (N-acetyl) transferase 4, core 2 | 0.0045 | −1.8965 |
| 3267036 | GRK5//G protein-coupled receptor kinase 5 | 0.0026 | −1.8544 |
| 3323052 | NAV2//neuron navigator 2 | 0.0045 | −1.8362 |
| 2778440 | UNC5C//unc-5 homolog C (C. elegans) | 0.0263 | 1.8141 |
| 3988435 | DOCK11//dedicator of cytokinesis 11 | 0.0200 | 1.8434 |
| 3797561 | LAMA1//laminin, alpha 1 | 0.0020 | 1.8583 |
| 2794584 | GPM6A//glycoprotein M6A | 0.0043 | 1.8642 |
| 3286602 | CXCL12//chemokine (C-X-C motif) ligand 12 | 0.0189 | 1.8834 |
| 3942472 | TCN2//transcobalamin II; macrocytic anemia | 0.0049 | 1.8857 |
| 2493858 | MAL//mal, T-cell differentiation protein | 0.0224 | 1.8862 |
| 2931036 | ULBP1//UL16 binding protein 1 | 0.0325 | 1.8913 |
| 2513554 | FAM130A2//family with sequence similarity 130, member A2 | 0.0089 | 1.9516 |
| 2913277 | KCNQ5//potassium voltage-gated channel, KQT-like subfamily, | 0.0177 | 1.9548 |
| | member 5 | | |
| 3127610 | PEBP4//phosphatidylethanolamine-binding protein 4 | 0.0269 | 2.0030 |
| 2931090 | PPP1R14C//protein phosphatase 1, regulatory (inhibitor) | 0.0093 | 2.0086 |
| | subunit 14C | | |
| 3198346 | PTPRD//protein tyrosine phosphatase, receptor type, D | 0.0417 | 2.0209 |
| 2773719 | CDKL2//cyclin-dependent kinase-like 2 (CDC2-related kinase) | 0.0183 | 2.0970 |
| 3327166 | C11orf74//chromosome 11 open reading frame 74 | 0.0020 | 2.1149 |
| 2434031 | HIST2H2BF//histone cluster 2, H2bf | 0.0023 | 2.1827 |
| 3831233 | (chr19: 36638840-36638939 (+), Len = 100) | 0.0058 | 2.2049 |
| 4007437 | SLC38A5//solute carrier family 38, member 5 | 0.0221 | 2.2177 |
| 3722355 | RND2//Rho family GTPase 2 | 0.0020 | 2.2972 |
| 2643592 | EPHB1//EPH receptor B1 | 0.0037 | 2.2986 |
| 3442150 | ACRBP//acrosin binding protein | 0.0008 | 2.3094 |
| 3985008 | TCEAL2//transcription elongation factor A (SII)-like 2 | 0.0407 | 2.3526 |
| 2443476 | SELE//selectin E (endothelial adhesion molecule 1) | 0.0149 | 2.6254 |
| 2686458 | ABI3BP//ABI gene family, member 3 (NESH) binding protein | 0.0141 | 3.2588 |

Clinical application of this technology, however, requires analysis of as many samples as possible with the minimum costs. This can be achieved by reducing the number of genes to be analyzed.

We used the principal component analysis to interrogate if it is possible to reduce the number of genes without loosing discrimination power. We found that differences in the expression levels of four of the 42 genes, EPHB1, GPM6A, ABI3BP and ANGPT2 (“4-gene signature”), correctly identifies most of the variation between samples (Table 3).

TABLE 3
Sample classification based on a 4-gene signature.

| | # per | # | # | % | % | Std. |
|---|---|---|---|---|---|---|
| | Class | Correct | Error | Correct | Error | Error |
| FC | 18 | 17 | 1 | 94.44 | 5.56 | 5.40 |
| NC | 24 | 20 | 4 | 83.33 | 16.67 | 7.61 |
| Total | 42 | 37 | 5 | 88.10 | 11.90 | 5 |
| Normalized | | | | 88.89 | 11.11 | |

FC = follicular thyroid carcinoma
NC = thyroid non-carcinoma (includes non-malignant tumors (follicular adenomas) and normal thyroid)

As we observed, the 4-gene signature correctly classifies 94.4% of the FC (1 out of 18 is classified as non-malignant) and 83% of the non-tumoral samples (4 out of 24 are incorrectly considered as malignant). By using those genes it was possible them to classify our group of 42 samples with 89% confidence (% correct).

Gene-Level Validation

Although microarray analysis is a very powerful tool to study gene-expression patterns, it is also very expensive and time-consuming. For these reasons, we took advantage of a different technique, real-time PCR, to validate our 4-gene signature. This technique consists of a modification of conventional PCR (polymerase chain reaction) that allows the operator to actually view the increase on the amount of product as it is being generated. Within the exponential phase, the real-time PCR instrument calculates two values. The Threshold line is the level of detection at which a reaction reaches a fluorescent intensity above background. The PCR cycle at which the sample reaches this level is called the Cycle Threshold, Ct. The Ct value is used in downstream quantitation or presence/absence detection. By comparing the Ct values of samples of unknown concentration with a series of standards, the amount of template DNA in an unknown reaction can be accurately determined.

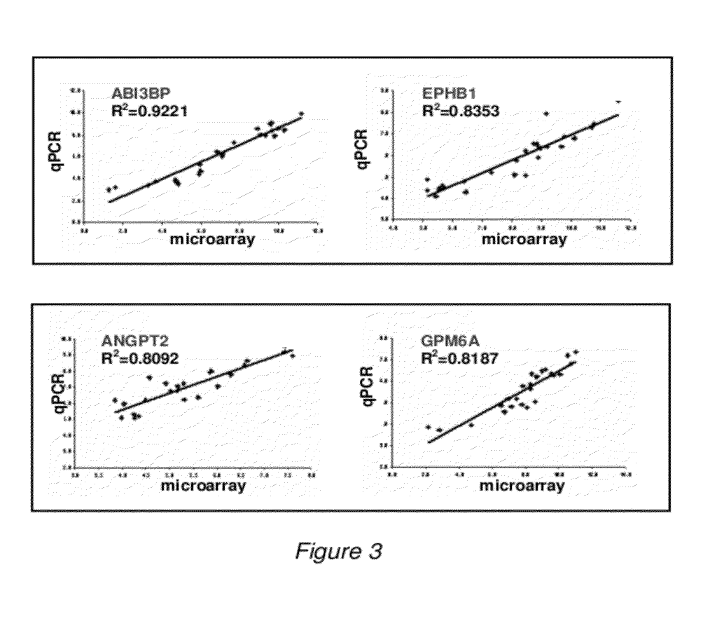

We used 24 from the 42 samples originally analyzed on the microarray study to validate the 4-gene signature by RT-qPCR with TaqMan probes. Each sample was amplified to detect the expression levels for each of the four genes plus four housekeeping (see Table 1). After calculating the ΔCt value, qPCR results (y-axis) were then individually plotted again the values obtained previously on the microarray (x-axis). As FIG. 3 shows, in all cases, a good correlation (R²>0.8) was obtained.

Next, a discriminant function was generated by using the SPSS statistic package. This function assigned each sample to a group, carcinoma or non-carcinoma, based on the qPCR data. By using this function, we were able to correctly classify 87.5% of the 24 samples tested by qPCR. This confirmed that quantitative PCR (qPCR) faithfully replicates the microarray findings.

The function we initially used employed the expression (ΔCt) from the 4 genes. Using a different kind of analysis we classified similarly the samples using only three genes (EPHB1, ABI3BP and ANGPT2) or even using only two of these genes (ABI3BP and ANGPT2).

Specifically, by using stepwise discriminant analysis it was possible to reduce the number of genes in the signature to three. We observed that a 3-gene signature (ABI3BP, ANGPT2 and EPHB1) was able to correctly classify 87.5% of the 24 samples tested by qPCR.

By using binary logistic regression it was possible to reduce the number of genes in the signature to two. We observed that a 2-gene signature (ABI3BP and ANGPT2) was able to correctly classify 87.5% of the 24 samples tested by qPCR.

Gene-Level Validation with External Samples

For a potential use of our 4-gene signature as a discriminator between malignant (carcinomas) and non-carcinomas, we should be able to apply it to any other group of samples unrelated to the ones used for establishing the discriminant function. To test that, we analyzed first an independent set of 12 samples from the Hospital Clinico Universitario de Santiago de Compostela (CHUS, Santiago de Compostela, Spain) using the qPCR methodology described above. These 12 samples were classified with 75% success using both the 4-gene and the 3-gene signature.

A second independent set of 19 samples was obtained from the Institute of Molecular Pathology and Immunology of the University of Porto (IPATIMUP, Porto, Portugal) and was analyzed using the qPCR methodology described above. In this case, the percentage of samples that were correctly assigned was 73.7% using both the 4-gene signature and the 3-gene signature.

Finally, we compared the power of discrimination obtained with the 4-gene signature against each one of the genes considered separately, by analyzing the results obtained with the qPCR analysis of the 12 external samples from the CHUS (Table 4).

TABLE 4
Discrimination power of the 4-gene signature and
the 3-gene signature vs. the individual genes.

| | % samples |
|---|---|
| classificator | correctly classified |
| 4-genes | 75.0 |
| 3-genes | 75.0 |
| ANGPT2 | 58.3 |
| ABI3BP | 58.3 |
| EPHB1 | 58.3 |
| GPM6A | 50.0 |

Altogether, our results show that quantification of gene expression level by the 4-gene signature and the 3-gene signature using qPCR was able to correctly classify three independent groups of samples with more than 70% reliability.

### Example 2

### Determination of FOLH1 Exon 1 Differential Expression in Benign Vs. Malignant Thyroid Tumors

Exon-Level Analysis

Besides whole gene expression, exon array technology makes possible to achieve a second analysis level, that is, exon-level. This analysis is focused on the detection of specific alterations in exons within a specific gene, by searching for differences on the signal obtained from individual probes. Because statistical analysis of exon information is complex and can lead to false positives, analysis was independently performed with three software packages, Partek Genomics Suite, EasyExon (Chang T Y et al, 2008) and OneChannelGUI (part of Bioconductor). Then we focused in a differentially expressed probe which was detected as significant by the three analyses.

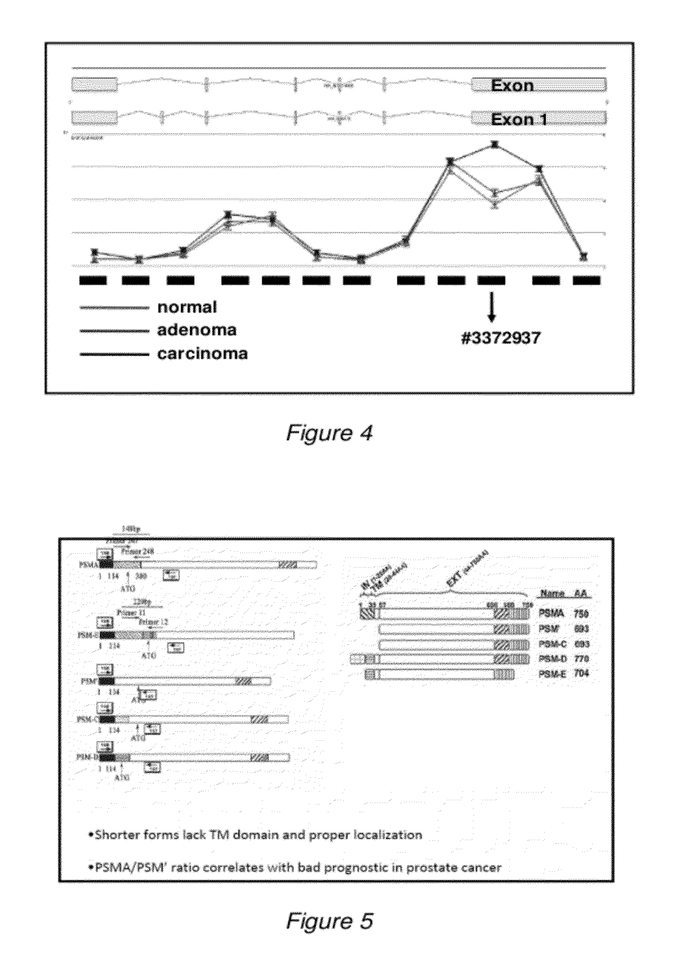

FIG. 4 shows the signals detected from all the probes within the array that are covering FOLH1 gene, after analysis with the Partek Genomics Suite analysis program. Signals from normal thyroid (green line), adenomas (red line) and carcinomas (blue line) obtained from the different probes (bottom rectangles) are always equivalent, except for the case of probe #3372937. This probe is located on FOLH1 exon 1 and clearly distinguishes between carcinomas and non-carcinomas or normal thyroid, suggesting that carcinomas are expressing a higher proportion of an RNA variant that includes exon 1 in relation to normal or adenoma samples.

FOLH1 gene (also known as PSMA, prostate-specific membrane antigen) is transcribed into different transcript variants that may or may not include exon 1 (FIG. 5). Full length forms, those that carry exon 1, are translated in a FOLH1/PSMA protein that is able to anchor cytoplasmic cell membrane, whereas a short form lacking exon 1 (PSM′) does not have this ability. It has been described that the ratio between both forms (long and short) is an indicator of bad prognosis in prostate tumors (Su et al, 1995; Schmittgen et al, 2003).

Expression of the full length (PSMA) and short (PSM′) forms on thyroid samples was first confirmed by conventional PCR, by subcloning and sequencing PCR products.

Exon-Level Validation

To analyze the FOLH1/PSM′ ratio, a qRT-PCR was performed. To detect PSMA (FOLH1) isoform a TaqMan® Gene Expression Assay was used, and to detect PSM′ isoform a Custom TaqMan® Gene Expression Assay that specifically recognized this splicing variant was generated. qRT-PCR reactions were performed on an ABI PRISM 7300 HT Sequence Detection System (Applied Biosystems) using TaqMan® Gene Expression Assays (Applied Biosystems). Assay reference numbers are listed in Table 1.

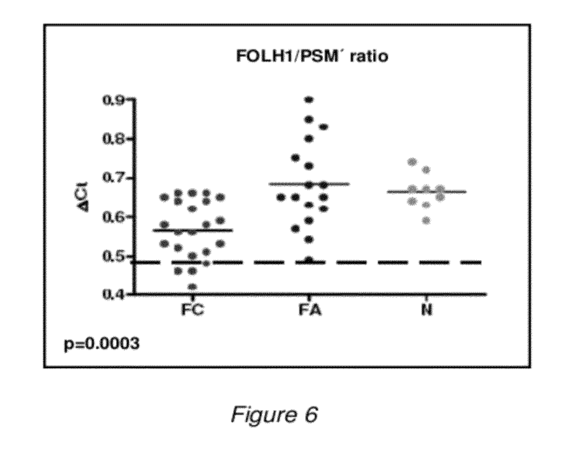

The Ct values for each transcript variant were corrected with the mean of the Ct values of the housekeeping genes to calculate the ΔCt value. Then, the ratio FOLH1/PSM′ was established (FIG. 6).

Carcinomas present a lower FOLH1/PSM′ ratio when compared with non-carcinoma samples. Although means between these two groups are statistically different, there is an overlapping region that ranges from 0.7 to 0.5. However, FOLH1/PSM′ratios lower than 0.5 (dashed line) are only present on adenocarcinomas.

Taken together, our results show that a FOLH1/PSM′ Ct ratio <0.5 can be used as a diagnostic marker for malignancy in follicular thyroid tumors and as a prognostic marker.

### Example 3

### Detection of Protein Levels

To provide for implementation of the diagnostic assay described herein in clinics, an feasible assay is developed to detect protein levels of the biomarkers. As a first step in the development of protein assays, experiments are performed to confirm at the protein level the findings described for the 4-gene signature. Commercial antibodies are available for the products of all four genes, such as: against GPM6A, polyclonal antibodies HPA017338 from Sigma-Aldrich (St. Louis, Mo.) or AP9341b from Abgent (San Diego, Calif.); against EPHB1: monoclonal antibody 3980S from Cell Signaling Technologies (Danvers, Mass.) or polyclonal antibody sc-28979 from Santa Cruz Biotechnologies (Santa Cruz, Calif.); against ABI3BP, monoclonal antibody H00025890-M15 from Novus Biologicals (Littleton, Colo.) or polyclonal antibody ab68612 from Abcam (Cambridge, Mass.); and against ANGPT2, monoclonal antibody CMA105 from Cell Sciences (Canton, Mass.) or polyclonal antibody PAB12280 from Abnova (Neihu District, Taipei City, Taiwan). Western blot, immunohistochemistry and/or ELISA assays are performed in thyroid tumour samples.

### REFERENCES

Barden C B, Shister K W, Zhu B, et al. “Classification of follicular thyroid tumors by molecular signature: results of gene profiling”. Clin Cancer Res. 2003; 9(5):1792-1800.
Cerutti J M, Delcelo R, Amadei M J, et al. “A preoperative diagnostic test that distinguishes benign from malignant thyroid carcinoma based on gene expression”. J Clin Invest. 2004; 113(8):1234-1242.
Eszlinger M, Wiench M, Jarzab B, et al. “Meta- and reanalysis of gene expression profiles of hot and cold thyroid nodules and papillary thyroid carcinoma for gene groups”. J Clin Endocrinol Metab. 2006; 91:1934-1942.
Eszlinger M and Paschke R. “Molecular fine-needle aspiration biopsy diagnosis of thyroid nodules by tumor specific mutations and gene expression patterns”. Mol Cell Endocrinol. 2010; 322: 29-37.
Faggiano A, Caillou B, Lacroix L, et al. “Functional characterization of human thyroid tissue with immunohistochemistry”. Thyroid. 2007; 7: 203-211.
Faquin W C. “Diagnosis and reporting of follicular-patterned thyroid lesions by fine needle aspiration”. Head Neck Pathol. 2009; 3: 2-5.
Finley D J, Zhu B, Barden C B, Fahey T J III. “Discrimination of benign and malignant thyroid nodules by molecular profiling”. Ann Surg. 2004; 240(3):425-437.
Foukakis T, Gusnanto A, Au A Y, et al. “A PCR-based expression signature of malignancy in follicular thyroid tumors”. Endocr Relat Cancer. 2007; 14:381-391.
Franc B, de la Salmoniere P, Lange F, et al. “Interobserver and intraobserver reproducibility in the histopathology of follicular thyroid carcinoma”. Hum Pathol. 2003; 34: 1092-1100.
Griffith O L, Melck A, Jones S J, Wiseman S M. “Meta-analysis and meta-review of thyroid cancer gene expression profiling studies identifies important diagnostic biomarkers”. J Clin Oncol. 2006; 24(31):5043-5050.
Huynh-Do U, Vindis C, Liu H, et al. “Ephrin-B1 transduces signals to activate integrin-mediated migration, attachment and angiogenesis”. J Cell Sci. 2002; 115: 3073-81.
Hodgson N C, Button J, Solorzano C C. “Thyroid cancer: is the incidence still increasing?”. Ann. Surg. Oncol. 2004; 11, 1093-1097.
Jarzab B, Wiench M, Fujarewicz K, et al. “Gene expression profile of papillary thyroid cancer: sources of variability and diagnostic implications”. Cancer Res. 2005; 65: 1587-1597.
Kondo T, Ezzat S, Asa S L. “Pathogenetic mechanisms in thyroid follicular-cell neoplasia”. Nat Rev Cancer. 2006; 6: 292-306.
Mazzanti C, Zeiger M A, Costouros N G, et al. “Using gene expression profiling to differentiate benign versus malignant thyroid tumors”. Cancer Res. 2004; 64: 2898-2903.
Mlcochová P, Barinka C, Tykvart J, et al. “Prostate-specific membrane antigen and its truncated form PSM′”. Prostate. 2009; 69:471-9.
Latini F R, Hemerly J P, Oler G, et al. “Re-expression of ABI3-binding protein suppresses thyroid tumor growth by promoting senescence and inhibiting invasion”. Endocr Relat Cancer. 2008; 15(3):787-99.
Lope V, Pollán M, Pérez-Gómez B, et al “Municipal mortality due to thyroid cancer in Spain”. BMC public health. 2006; 6:302-311.
Real-Time PCR versus Traditional PCR. Applied Biosystems Tutorial.
Schmittgen T D, Teske S, Vessella R L, et al. “Expression of prostate specific membrane antigen and three alternatively spliced variants of PSMA in prostate cancer patients”. Int J Cancer. 2003; 107: 323-329.
Sheng Z, Wang J, Dong Y, et al. “EphB1 is underexpressed in poorly differentiated colorectal cancers”. Pathobiology. 2008; 75: 274-80.
Srirajaskanthan R, Dancey G, Hackshaw A, et al. “Circulating angiopoietin-2 is elevated in patients with neuroendocrine tumours and correlates with disease burden and prognosis”. Endocr Relat Cancer. 2009; 16(3):967-76.
Su S L, Huang I P, Fair W R, et al. “Alternatively spliced variants of prostate-specific membrane antigen RNA: ratio of expression as a potential measurement of progression”. Cancer Res. 1995; 55:1441-1443.
Wang J D, Dong Y C, Sheng Z, et al. “Loss of expression of EphB1 protein in gastric carcinoma associated with invasion and metastasis”. Oncology. 2007; 73: 238-45.
Weber F, Shen L, Aldred M A, et al. “Genetic classification of benign and malignant thyroid follicular neoplasia based on a 3-gene combination”. J Clin Endocrinol Metab. 2005; 90: 2512-2521.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety, particularly for the teaching referenced herein.

1. A method of diagnosing follicular thyroid cancer in a human comprising
contacting a biological sample from a subject with reagents that specifically bind to a panel of biomarkers comprising ABI3BP and ANGPT2, and
determining whether the biomarkers are differentially expressed in the sample relative to a control; thereby diagnosing follicular thyroid cancer.

2. The method of claim 1, wherein the panel of biomarkers comprises EPHB1, ABI3BP and ANGPT2 or EPHB1, GPM6A, ABI3BP and ANGPT2.

3. The method of claim 1, wherein the sample is a biopsy.

4. The method of claim 3, wherein the biopsy is a fine needle aspiration biopsy.

5. The method of claim 1, wherein the reagent is a nucleic acid.

6. The method of claim 5, wherein the reagent is an oligonucleotide or an RT PCR primer set.

7. The method of claim 1, wherein the reagent is an antibody or an antigen-binding fragment thereof.

8. The method of claim 7, wherein the antibody is a monoclonal antibody.

9. The method of claim 1, wherein the expression of ANGPT2 is upregulated relative to a control and wherein the expression of EPHB1, GPM6A and/or ABI3BP is down-regulated relative to a control.

10. The method of claim 1, wherein the differential expression in the sample relative to the control is at least 1.8 fold.

11. The method of claim 1, wherein the control is a level of expression determined in a non-malignant thyroid tissue sample.

12. The method of claim 1, wherein the panel of biomarkers consists of (1) ABI3BP and ANGPT2; (2) EPHB1, ABI3BP and ANGPT2; or (3) EPHB1, GPM6A, ABI3BP and ANGPT2.

13. A kit comprising reagents that specifically bind to a panel of biomarkers comprising ABI3BP and ANGPT2.

14. The kit of claim 13, wherein the panel of biomarkers comprises EPHB1, ABI3BP and ANGPT2 or EPHB1, GPM6A, ABI3BP and ANGPT2.

15. The kit of claim 13, wherein the reagents comprise one or more nucleic acids or one or more antibodies or antigen-binding fragments thereof.

16. The kit of claim 15, wherein the nucleic acids comprise one or more oligonucleotides.

17. The kit of claim 16, wherein the oligonucleotides comprise RT-PCR primers.

18. The kit of claim 15, wherein the one or more antibodies is one or more monoclonal antibodies.

19. The kit of claim 13, wherein the reagents are detectably labeled or wherein the kit further comprises one or more detectable labels.

20. The kit of claim 13, wherein the panel of biomarkers consists of (1) ABI3BP and ANGPT2; (2) EPHB1, ABI3BP and ANGPT2; or (3) EPHB1, GPM6A, ABI3BP and ANGPT2.

21. A method of diagnosing thyroid adenocarcinoma in a human comprising
contacting a biological sample from a subject with reagents that specifically bind to full length FOLH1 and FOLH1 lacking exon 1 (PSM′),
determining expression of FOLH1 and PSM′ in the sample, and
determining a FOLH1/PSM′ ratio of expression; wherein a FOLH1/PSM′ ratio lower than 0.5 indicates that the subject has a thyroid adenocarcinoma.

22. A kit comprising reagents that specifically bind to FOLH1 and FOLH1 lacking exon 1 (PSM′).
